(19) United States
(12) Patent Application Publication
(10) Pub. No.: US 2008/0161799 A1
(43) Pub. Date: Jul. 3, 2008

(54) Position independent catheter

(76) Inventors: Todd Stangenes, Minneapolis, MN (US); Dale Just, Minneapolis, MN (US)

Correspondence Address: SJM/AFD-WILEY, 14901 DEVEAU PLACE, MINNETONKA, MN, 55345-2126, US

(21) Appl. No.: 11/647,317

(22) Filed: Dec. 29, 2006

(51) Int. Cl. A61B 18/18

(52) U.S. Cl. 606/41

(57) ABSTRACT

A catheter for treating tissue is disclosed, the catheter having slotted openings on a circumference of its distal portion, wherein each slotted opening spans at least about 25% of the circumference of the catheter. The disclosed catheter is capable of treating a variety of tissue in a variety of configurations. For example, in an ablation catheter of the disclosed invention, the slotted openings on the distal portion allow the ablation catheter to create effective lesions in both the pulmonary veins and the posterior wall of the left atrium. The ablation catheter preferably carries a conductive medium to help deliver ablation energy (e.g., RF) to the tissue being ablated. The configuration of the catheter permits ablation to be conducted in many varieties and geometric orientations independently of the tissue orientation. Also disclosed is a method of ablating tissue using catheter with slotted openings.

### BACKGROUND OF THE INVENTION

a. Field of the Invention

The instant invention relates to catheters. In particular, the instant invention relates to cardiac catheters, and further relates to catheters that are especially suited to ablation of cardiac tissue. The catheters of the present invention comprise slotted openings at their distal end, each slotted opening is arranged around the circumference of the catheter shaft and covers at least about 25% of the circumference of the catheter shaft. These slotted ablation catheters allow for effective ablation lesions to be created in a wide variety of cardiac tissue orientation.

b. Background Art

Catheters are commonly used by physicians to perform a wide variety of medical procedures on various locations in the body that would otherwise be inaccessible without more invasive procedures. In the cardiac field, catheters are frequently used for angioplasty (to open clogged blood vessels), to ablate cardiac tissue to help restore a more regular heartbeat in arrhythmia patients, or to monitor various electrical activity. Such cardiac catheters are typically tubular in shape and may comprise various electrodes, wires, optical fibers, and/or passageways for carrying and delivering fluid to cardiac tissue, depending on the purpose for which the catheter is meant.

As illustrated in FIG. 1, a typical human heart 30 includes a right ventricle 98, a right atrium 94, a left ventricle 100 and a left atrium 104. The right atrium is in fluid communication with the superior vena cava 92 and the inferior vena cava 96. The interatrial septum separates the right atrium from the left atrium. The tricuspid valve contained within the atrioventricular septum provides a fluid flow path between the right atrium with the right ventricle. On the inner wall of the right atrium where it is connected with the left atrium is a thin walled, recessed area, referred to as the fossa ovalis. Between the fossa ovalis and the tricuspid valve is the opening or ostium for the coronary sinus. The coronary sinus is the large epicardial vein which accommodates most of the venous blood which drains from the myocardium into the right atrium.

In a normally functioning heart, contraction and relaxation of the heart muscle (myocardium) takes place in an organized fashion as electrochemical signals pass sequentially through the myocardium from the sinoatrial (SA) node (not shown) located in the right atrium to the atrialventricular (AV) node (not shown) and then along a well defined route which includes the His-Purkinje system into the left and right ventricles. Initial electric impulses are generated at the SA node and conducted to the AV node. The AV node lies near the ostium of the coronary sinus in the interatrial septum in the right atrium. The His-Purkinje system begins at the AV node and follows along the membranous interatrial septum toward the tricuspid valve through the atrioventricular septum and into the membranous interventricular septum. At about the middle of the interventricular septum, the His-Purkinje system splits into right and left branches which straddle the summit of the muscular part of the interventricular septum.

Arrhythmia is an abnormal heart rhythm that occurs in some individuals. Certain types of arrhythmia lead to significant patient discomfort and even death. Pathological causes for some arrhythmias are difficult to diagnose, but are believed to be due to stray circuits within the left and/or right atrium of the heart. These circuits or stray electrical signals are believed to interfere with the normal electrochemical signals passing from the SA node to the AV node and into the ventricles. Efforts to alleviate these problems in the past have included significant usage of various drugs. In some circumstances drug therapy is ineffective and frequently is plagued with side effects such as dizziness, nausea, and vision problems.

An increasingly common medical procedure for the treatment of certain types of arrhythmia involves the ablation of tissue in the heart to cut off the path for stray or improper electrical signals. Such procedures are typically performed with an ablation catheter. Typically, the ablation catheter is inserted in an artery or vein in the leg, neck, or arm of the patient and threaded, sometimes with the aid of a guidewire or introducer, through the vessels until a distal tip of the ablation catheter reaches the desired location in the heart. The ablation catheters commonly used to perform these ablation procedures produce lesions in cardiac tissue. The resulting lesions electrically isolate or render the tissue non-contractile at particular points. The lesion partially or completely blocks the stray electrical signals to lessen or eliminate atrial fibrillations.

The energy necessary to ablate cardiac tissue and create a permanent lesion can be provided from a number of different sources. Direct current through a laser, microwave, ultrasound, and other forms of energy have been utilized to perform ablation procedures. Because of problems associated with the use of DC current, however, radiofrequency (RF) has become one of the preferred sources of energy for ablation procedures. In addition to radiofrequency ablation catheters, thermal ablation catheters have been disclosed. During thermal ablation procedures, a heating element, secured to the distal end of a catheter, heats thermally conductive fluid, which fluid then contacts the human tissue to raise its temperature for a sufficient period of time to ablate the tissue.

In some conventional ablation procedures, the ablation catheter includes a single distal electrode secured to the tip of the ablation catheter to produce small lesions wherever the tip contacts tissue. To produce a linear lesion, the tip may be dragged slowly along the tissue during energy application. Increasingly, however, cardiac ablation procedures utilize multiple electrodes affixed to the catheter body to form multiple lesions.

One difficulty in obtaining an adequate ablation lesion using conventional ablation catheters is the constant movement of the heart, especially when there is an erratic or irregular heart beat. Another difficulty in obtaining an adequate ablation lesion is caused by the inability of conventional catheters to obtain and retain uniform contact with the cardiac tissue across the entire length of the ablation electrode surface. Without such continuous and uniform contact, any ablation lesions formed may not be adequate.

Another difficulty encountered with existing ablation catheters is assurance of adequate tissue contact. Current techniques for creating continuous linear lesions in endocardial applications include, for example, dragging a conventional catheter on the tissue, using an array electrode, or using pre-formed electrodes. These catheter designs either require significant technical skill on the part of the physician in guiding and placing the catheter by sensitive steering mechanisms. Further, all of these devices comprise rigid electrodes that do not always conform to the tissue surface, especially when sharp gradients and undulations are present, such as at the ostium of the pulmonary vein in the left atrium and the isthmus of the right atrium between the inferior vena cava and the tricuspid valve. Consequently, continuous linear lesions are difficult to achieve. A need exists for an improved catheter, particularly, a catheter design that achieves cardiac ablation effectively and independently of the orientation of target tissue. A need also exists for a catheter that addresses the vast anatomical differences found in the heart, especially, the left atrium and pulmonary veins.

### BRIEF SUMMARY OF THE INVENTION

The present invention relates to a catheter for treating tissue. The catheter of the present invention comprises at least one slotted opening, spanning at least 25% of the circumference of the catheter, perpendicular to the axis of the catheter, at the catheter's distal end. Energy, heat, fluids and/or medicaments may be supplied through at least one slotted opening to treat tissue proximal or distal to such slotted opening.

One object of the disclosed invention is to provide an improved ablation catheter for forming linear lesions in tissue, including tissue within the human heart and the pulmonary veins. This and other objects are provided by the ablation catheter that is disclosed by the present invention. The ablation catheter of the present invention is capable of making improved lesions in a variety of tissue. The ablation catheter is particularly useful for ablating cardiac tissue regardless of its orientation. The distal portion of the ablation catheter comprises at least one slotted opening, the slotted opening spanning at least 25% of the circumference of the catheter. The ablation catheter is adapted to provide RF energy, conductive fluid, and/or heat through the slotted opening(s), to proximal or distal cardiac tissue. As such, the catheter may be used to ablate, for example, both the pulmonary veins and the left atrium. In some embodiments of the present invention, the ablation catheter has a curved distal end.

Disclosed herein is an ablation catheter for ablating tissue. The ablation catheter includes a catheter shaft having a proximal portion and a distal portion. The distal portion is adapted to be inserted into a body having tissue to be treated and is disposed remotely from the proximal portion. The distal portion includes a plurality of slotted openings located on a circumference of the distal portion. The slotted openings are adapted to deliver conductive fluid to the tissue to be ablated, and the plurality of slotted openings are arranged along the circumference of the catheter shaft (perpendicular to the main axis of the catheter), such that each slotted opening spans at least about 25% of the circumference of the distal portion of the catheter shaft. The ablation catheter also includes a lumen disposed within the distal portion, and the lumen is adapted to carry a conductive medium (e.g., saline). The ablation catheter also includes an electrode disposed within the distal portion of the catheter shaft, which electrode is adapted to supply ablation energy to the conductive fluid. The ablation catheter may optionally include a fluid manifold along at least a portion of the electrode. The fluid manifold may include tubing made of polyvinyl alcohol foam, expanded polytetrafluoroethylene, or a combination thereof. Optionally, the distal portion includes one or more curved sections, which may be created using one or more shape memory wires. In one configuration, the slotted openings may span about 33% of the circumference of the distal portion of the catheter shaft.

Also disclosed herein is a catheter for treating tissue, the catheter comprising a catheter shaft that has a proximal portion and a distal portion. The distal portion is adapted to be inserted into a body having tissue to be treated and has at least one slotted opening located on a circumference of the distal portion. The slotted opening is adapted to introduce a therapeutic energy, heat, fluid, or medicament to the tissue to be treated. The slotted opening is arranged perpendicular to the axis of the distal portion and spans about one third to about two thirds of the circumference of the distal portion of the catheter shaft. The distal portion of the catheter may optionally have at least one lumen adapted to carry a conductive medium. The distal portion of the catheter may also optionally have an electrode and a conductive medium manifold running along a portion of the electrode, the conductive medium manifold having a plurality of passage ways through which a conductive medium may pass. The distal portion of the catheter may also optionally have a lumen adapted to carry a conductive medium from the proximal portion to at least one slotted opening, and a metal electrode mounted within the lumen, wherein the metal electrode is adapted to supply ablation energy to the conductive medium. The metal electrode may be a platinum flat wire adapted to be connected to an RF generator by an electrical lead that extends through at least a portion of the distal portion of the catheter shaft. The disclosed catheter may have at least one curved section at its distal portion. The disclosed catheter may further have a shape memory wire at its distal portion. The shape memory wire may be located within a second lumen extending along the distal portion of the catheter shaft.

Also disclosed herein is an ablation catheter for ablating tissue, the ablation catheter having a catheter shaft having a proximal portion and a distal portion. The distal portion of the ablation catheter is adapted to be inserted into a body having tissue to be treated. The distal portion has a plurality of slotted openings located on a circumference of the distal portion, each slotted opening spanning at least about 90° of the circumference of the distal portion. The ablation catheter also has a metal electrode disposed within the distal portion, the electrode being adapted to supply ablation energy through the slotted openings to the tissue to be ablated. In one configuration, the ablation catheter also has a fluid manifold along at least a portion of the metal electrode and the slotted openings are adapted to deliver conductive fluid to the tissue to be ablated. The fluid manifold may have tubing made of a porous polymer. The slotted openings of the ablation catheter may span about 120° to about 240° of the circumference of the distal portion.

Disclosed herein are also methods for treating cardiac arrhythmia. In one such method, an ablation catheter is inserted into a patient having cardiac tissue to be treated. The ablation catheter has a proximal portion and a distal portion, the distal portion comprising a plurality of slotted openings, adapted to introduce ablative energy to the cardiac tissue to be treated. The slotted openings of the ablation catheter are located on a circumference of the distal portion and span between about 90° and about 270° of the circumference of the catheter shaft. The ablation catheter also has an electrode disposed within the distal portion, the electrode has a fluid manifold along at least a portion of the electrode, and the electrode is adapted to be connected to an ablative energy source. The plurality of slotted openings permit the catheter to ablate tissue in both the posterior wall of the left atrium and the pulmonary vein. The ablation catheter is placed along the catheter tissue to be treated. An ablative energy is applied to the ablation catheter to form lesions on the cardiac tissue.

In another disclosed method, tissue that is in at least two different orientations within a body is simultaneously ablated using an ablation catheter of the present invention. The ablation catheter is inserted into a patient having tissue to be ablated, the ablation catheter having a proximal portion and a distal portion, the distal portion having a plurality of slotted openings. The slotted openings are adapted to introduce ablative energy to the tissue to be treated and are located on a circumference of the distal portion. Each slotted opening spans between about 33% and about 67% of the circumference of the catheter shaft. The ablation catheter has an electrode disposed within the distal portion, the electrode is adapted to be connected to an ablative energy source. The ablation catheter is placed along the tissue to be treated and ablative energy is applied to form lesions simultaneously on tissue that is in at least two different orientations.

The catheters of the present invention, with the slotted openings on their distal portions, are effective for treating a wide variety of tissue in a wide variety of orientations. The ablation catheter of the present invention can simultaneously create lesions in pulmonary veins and on atrial walls. The ablation catheter of the present invention is also effective for simultaneously creating lesions on the posterior wall of the left atrium and in the pulmonary veins. The catheters of the present invention thus save treatment time because the catheter does not have to be rearranged between treatments and one catheter accommodates vast anatomical differences in various tissue surfaces, especially in various cardiac surfaces.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a partial cut away diagram of a human heart.

FIG. 2 is an isometric view of an ablation catheter assembly according to the present invention.

FIG. 3A is a fragmentary view of a distal portion of a catheter.

FIG. 3B is a fragmentary view of a distal portion of another catheter.

FIG. 3C is a fragmentary view of a distal portion of a catheter according to the present invention.

FIG. 3D is a fragmentary view of a distal portion of a catheter according to the present invention.

FIG. 4A is a cross-sectional view of the distal portion of the catheter depicted in FIG. 3A.

FIG. 4B is a cross-sectional view of the distal portion of the catheter depicted in FIG. 3B.

FIG. 4C is a cross-sectional view of the distal portion of the catheter depicted in FIG. 3C.

FIG. 4D is a cross-sectional view of the distal portion of the catheter depicted in FIG. 3D.

FIG. 5A is a fragmentary view of the distal portion of a curved ablation catheter, looking perpendicular to the longitudinal axis of the catheter shaft.

FIG. 5B is a fragmentary view of the distal portion of the ablation catheter depicted in FIG. 5A, looking from the top of the distal portion down the longitudinal axis of the catheter shaft.

FIG. 6A is a fragmentary view of the distal portion of another curved ablation catheter, looking perpendicular to the longitudinal axis of the catheter shaft.

FIG. 6B is a fragmentary view of the distal portion of the ablation catheter depicted in FIG. 6A, looking from the top of the distal portion down the longitudinal axis of the catheter shaft.

FIG. 7A is a fragmentary view of the distal portion of a curved ablation catheter according to the present invention, looking perpendicular to the longitudinal axis of the catheter shaft.

FIG. 7B is a fragmentary view of the distal portion of the ablation catheter depicted in FIG. 7A, looking from the top of the distal portion down the longitudinal axis of the catheter shaft.

FIG. 8 is an isometric view of the ablation catheter depicted in, for example, FIGS. 7A and 7B, creating lesions on opposing vessel walls.

FIG. 9 is an isometric view of a heart with portions of the atria and ventricles broken away to reveal positioning of the ablation catheter depicted in, for example, FIGS. 7A and 7B, creating lesions in the left atrium.

FIG. 10 is an isometric view of a heart with portions of the atria and ventricles broken away to reveal positioning of the ablation catheter depicted in FIG. 9 creating lesions in the left superior pulmonary vein

FIG. 11 is an isometric view of a heart with portions of the atria and ventricles broken away to reveal position of the ablation catheter depicted in FIG. 9 creating lesions in both the left atrial wall and the left superior pulmonary vein simultaneously.

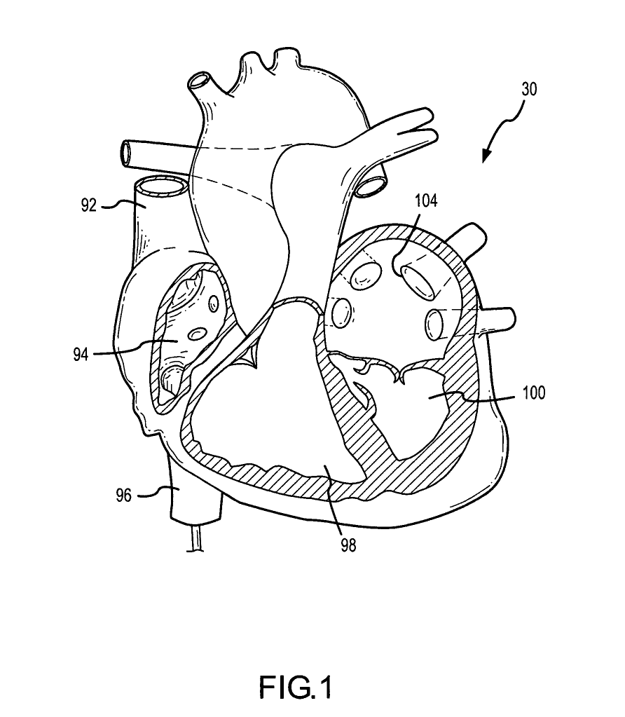

### DETAILED DESCRIPTION OF THE INVENTION

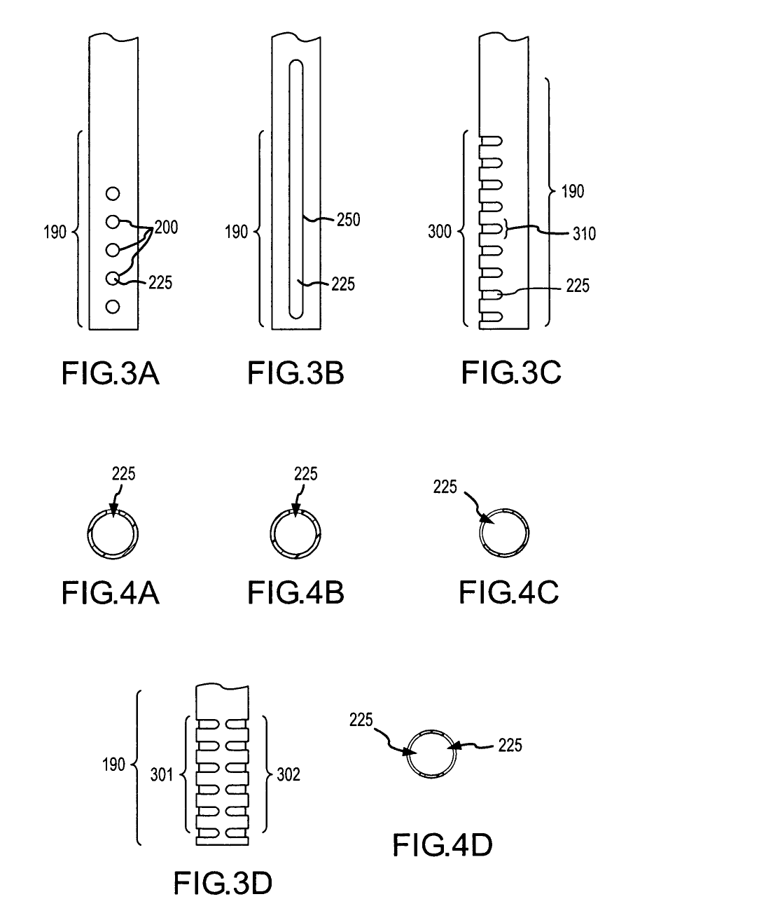

In general, the instant invention relates to a catheter for treating tissue. As is well known in the art, the catheter of the present invention has a catheter shaft with a proximal portion and a distal portion. FIGS. 3A and 3B show configurations for the distal portion of catheters known in the art. As is shown in FIG. 3A, the distal portion 190 of some catheters currently in use has a plurality of portholes 200 giving access to the inside of the catheter 225. Depending on the use for which such catheter is intended, the inside of the catheter may comprise a lumen for carrying conductive media or various medicaments, an electrode, a manifold, or any other equipment or substances necessary for treating tissue the catheter is intended to treat. Energy, heat, and/or medicaments may be supplied through the slotted openings to treat tissue proximal to such slotted opening. FIG. 3B shows another configuration for the distal portion 190 of a catheter known in the art. In the configuration of FIG. 3B, the distal portion 190 has a vertical slit 250 running parallel to the axis of the catheter shaft and exposing the inside of the catheter 225. FIGS. 4A and 4B further show a cross section of the known catheter configurations shown in FIGS. 3A and 3B.

FIG. 3C shows one embodiment of a catheter of the present invention. The distal portion 190 of the catheter shaft has at least one slotted opening 300 located on a circumference of the distal portion of the catheter. The slotted opening 300 spans at least about 25% (i.e., about 90°) of the catheter circumference. In one preferred embodiment, the distal portion comprises a plurality of slotted openings 300 and each slotted opening spans approximately 33% (i.e., about 120°) of the catheter circumference. The slotted openings 300 may span up to about two thirds (about 240°) of the catheter circumference, three quarters (270°) of the catheter circumference, or more. Preferably, the slotted openings will not affect the structural integrity of the catheter shaft. As illustrated in FIG. 3C, however, the present invention provides a catheter that can be used to treat multiple tissue surfaces having multiple orientations. This is a significant advantage over the prior art.

FIG. 3D shows another embodiment of a catheter of the present invention. The distal portion 190 has a plurality of sets of slotted openings 301 and 302 located on a circumference of the distal portion of the catheter. Each slotted opening spans at least about 25% of the circumference of the catheter. As illustrated in FIG. 3D, however, the present invention provides [symbol] invention may be made of a variety materials. In a preferred embodiment of the ablation catheter of the present invention, catheter comprises a metal electrode (e.g., platinum). In a further preferred embodiment, the electrode is a platinum flat wire adapted to be connected to an RF generator by an electrical lead that extends through at least a portion of the distal portion of the catheter shaft.

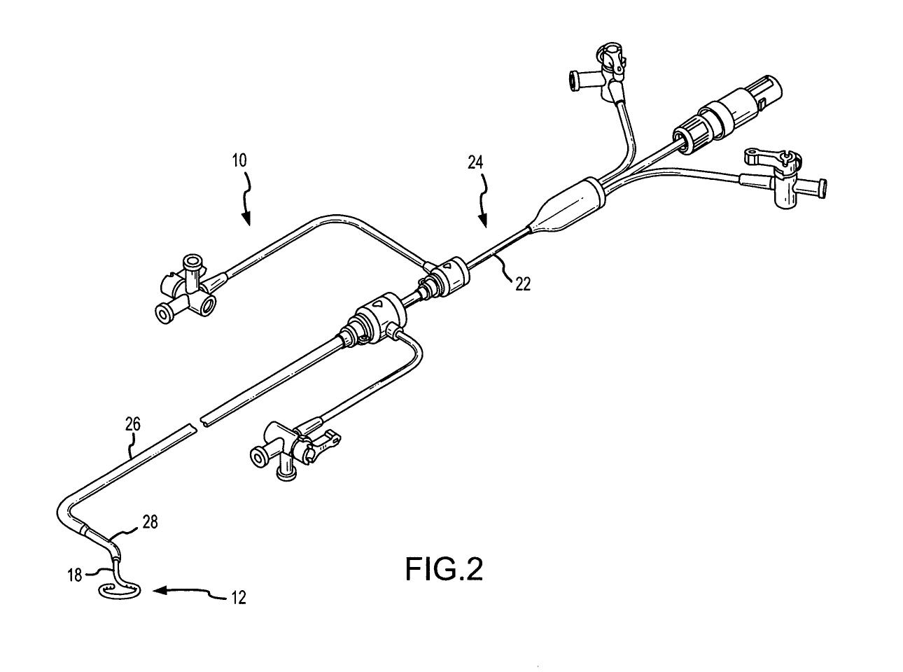

FIG. 2 is an isometric view looking downwardly at an ablation catheter assembly 10 according to the present invention. In this embodiment of the catheter assembly 10, an ablation catheter 18 comprising a catheter shaft 22 having a proximal portion 24 and a distal portion 12 is used in combination with one or more guiding introducers 26, 28 to facilitate formation of lesions on tissue, for example, cardiovascular tissue. As depicted in FIG. 2, the ablation catheter 18 may be used in combination with an inner guiding introducer 28 and an outer guiding introducer 26. Alternatively, a single guiding introducer may be used or a precurved transeptal sheath may be used instead of one or more guiding introducers. In general, the introducer, introducers, or precurved sheath are shaped to facilitate placement of the ablation catheter 18 at the tissue to be ablated. Thus, for example, the introducer or the introducers or the transeptal sheath make it possible to navigate to the heart and through its complex physiology to reach specific tissue to be ablated. When the ablation catheter 18 has a specific configuration like the curved configuration depicted in FIGS. 2, 5A, 5B, 6A, 6B, 7A, and 7B, the shape of the introducers 26, 28, if used, may change somewhat when the distal portion 12 of the ablation catheter 18 is retracted into the introducers 26, 28. A conductive fluid medium (e.g., hypertonic saline) contacting the electrode and the tissue to be ablated may comprise a virtual electrode, eliminating the need for direct contact between the electrode and the tissue to be ablated.

As further described in U.S. Pat. Nos. 7,122,034 and 7,101,362, and U.S. patent application Ser. No. 11/328,565, (all of which are incorporated herein by reference in their entireties) curved configuration catheters preferably have a catheter shaft comprising at least one curved section. The curved section may be formed by a memory wire within a lumen disposed within the catheter. Such catheters may comprise dual lumen systems to separate the memory wire from an electrode or multiple electrodes.

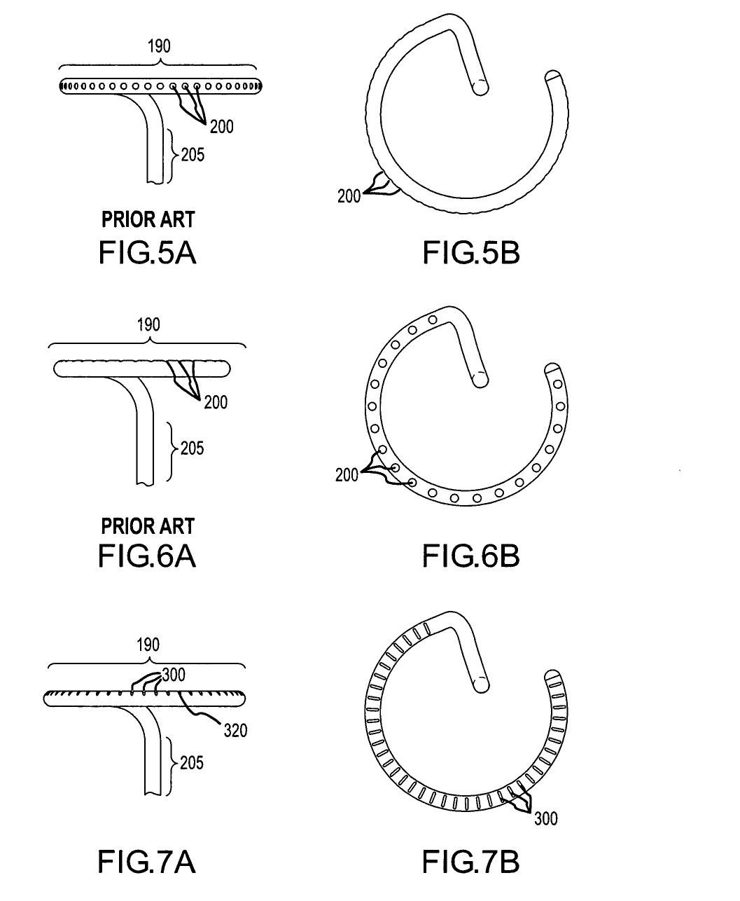

FIG. 5A depicts an embodiment of a known ablation catheter with a curved configuration at its distal end. The distal portion 190 of this catheter comprises a plurality of portholes 200 along the length of the curved distal portion 190, the portholes 200 face outward and perpendicular to the axis 205 of the catheter shaft. FIG. 5B shows the catheter of FIG. 5A viewed from the top of the distal portion down the longitudinal axis of the catheter shaft, showing the portholes 200 in outward alignment from the curved distal portion 190.

FIG. 6A depicts an embodiment of another known ablation catheter with a curved configuration at its distal end. The distal portion 190 of this catheter comprises a plurality of portholes 200 along the length of the curved distal portion 190, the portholes 200 facing upward, or forward, along the axis 205 of the catheter shaft. FIG. 6B shows the catheter of FIG. 6A viewed from the top of the distal portion down the longitudinal axis of the catheter shaft, showing the portholes 200 in upward alignment on the curved distal portion 190.

The present invention is effective for simultaneously creating lesions in pulmonary veins and on atrial walls. The present invention is also effective for simultaneously creating lesions in the posterior wall of the left atrium and in the pulmonary veins.

FIG. 7A depicts an embodiment of an ablation catheter according to the present invention. The curved distal portion 190 comprises at least one slotted opening 300. The slotted opening 300 spans at least about 25% of the circumference of the catheter and may be located at any point along the circumference of the catheter shaft. In one preferred embodiment, the distal portion 190 of the catheter comprises a plurality of slotted openings, each slotted opening spanning about 33% of the circumference of the catheter. In a further preferred embodiment, as shown in FIG. 7A, the plurality of slotted openings cover at least a portion of the upper forward surface of the catheter shaft and at least a portion of the outward surface of the catheter shaft. The slotted openings of the ablation catheters of the present invention create expanded access to the electrode 350 within the catheter from a variety of geometries. The configurations of the curved ablation catheters of the present invention are thus able to treat a variety of tissue regardless of tissue orientation. These novel curved ablation catheters with slotted openings can effectively form lesions in, for example, both the pulmonary veins and the posterior wall of the left atrium.

In one embodiment of the ablation catheter of the present invention, as shown in FIG. 7A, the slotted openings 300 have square ends 320, but the slotted openings 300 may also have rounded ends or ends of any other functional geometry. The slotted openings 300 may also be of any desired width and may span two thirds of the catheter circumference or more. Preferably, the slotted openings are configured to not affect the structural integrity of the catheter shaft. In one preferred embodiment of the present invention, the novel catheter has a distal end with a plurality of slotted openings, 300, the slotted openings 300 have rounded ends and may be preferably from about 0.5 mm to about 3.0 mm in width. The slotted openings 300 on the distal portion 190 of the catheter may each be of equal arc length and width or may have differing arc lengths and/or widths.

The catheter shaft of the curved ablation catheter of the present invention may be made of a variety of materials, including without limitation, polymeric materials such as PELLETHANE, polypropylene, oriented polypropylene, polyethylene, crystallized polyethylene terephthalate, polyethylene terephthalate, polyester, and polyvinyl chloride. The distal portion of the curved ablation catheter may also have only one curved portion or may have a plurality of curved portions to form, for example, a “C” shape or a circular shape.

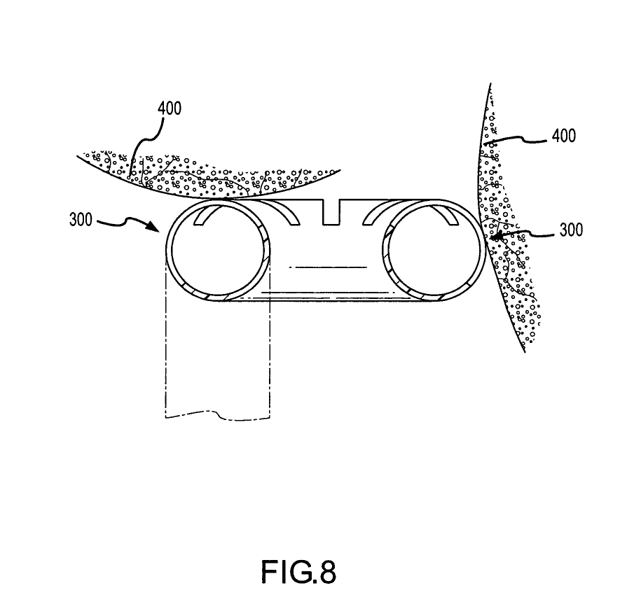

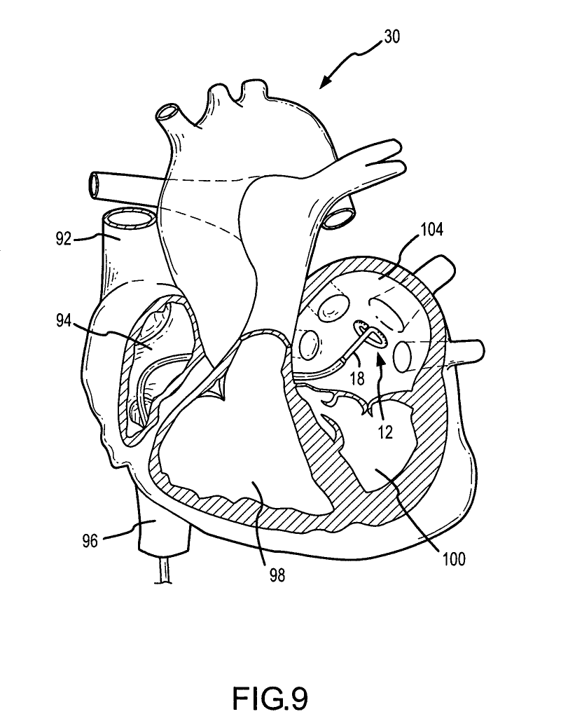

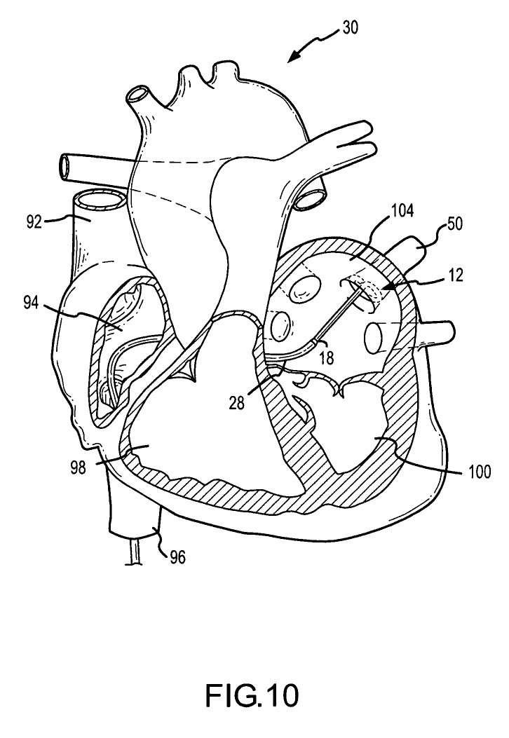

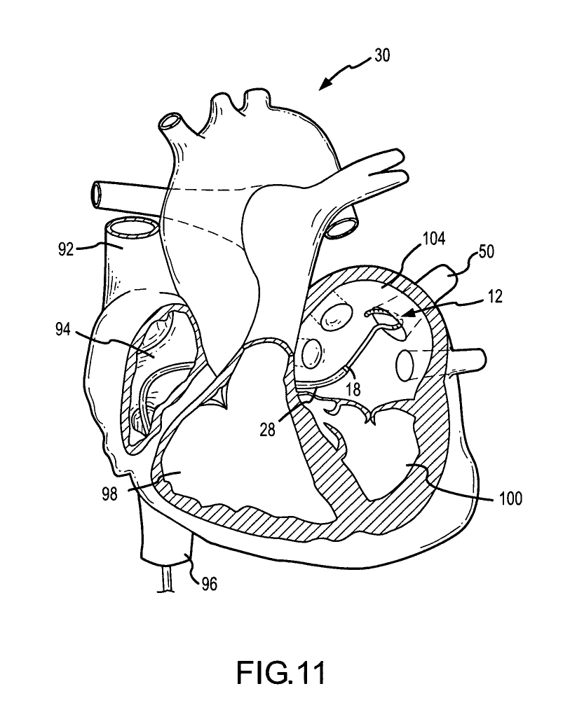

As shown in FIGS. 8-11, the curved ablation catheters of the present invention may be used to treat cardiac arrhythmias. In one embodiment of such a method of the present invention, as shown in FIG. 10, the distal portion 12 of the ablation catheter 18 has been inserted into the left superior pulmonary vein 50. While the ablation catheter 18 is in the pulmonary vein, the electrode would be activated to create the desired lesion in the left superior pulmonary vein 50. FIG. 9 shows the same ablation catheter as depicted in FIG. 10 being used to form lesions on the posterior wall of the left atrium 104. FIG. 11 further shows the same ablation catheter forming lesions on the posterior wall of the left atrium 104 and on the left superior pulmonary vein 50 at the same time. FIG. 8 shows the same ablation catheter as depicted in FIGS. 9 and 10, forming lesions on vessel walls 400 and 410 of varied geometry. FIG. 8 illustrates the ability of the present invention to address the vast anatomical differences in the cardiac area. The present invention allows for these anatomical differences because the electrode will function effectively regardless of the orientation of the tissue. As illustrated in FIG. 8, the present invention can be used to ablate tissue in multiple regions that are oriented differently, and the invention can do so with a single treatment. For example, as shown in FIG. 8, the ablation catheter of the present invention can treat a vertical vessel wall 410 and a horizontal vessel wall 400 at the same time. This constitutes a significant advantage over the prior art.

In one preferred embodiment, an RF electric current emanating from a metal electrode disposed within the catheter passes through the conductive fluid medium (e.g., saline) contained in a lumen in the catheter through the slotted openings and into the adjacent tissue. The conductive fluid medium may experience ohmic heating as it flows along the metal electrode and out the slotted openings. Ablation energy is delivered to the tissue via the conductive medium. Thus, a lesion is formed in the tissue by the RF energy. Lesion formation may also be facilitated by the conductive fluid medium, which may have been heated by ohmic heating to a sufficiently high temperature to facilitate or enhance lesion formation, flowing out the slotted openings. While the RF energy is being conducted into the adjacent tissue, the heated conductive fluid medium convectively affects the temperature of the tissue. In order to form a sufficient lesion, it is desirable to raise the temperature of the tissue to at least 50° C. for an appropriate length of time (e.g., one minute). Thus, sufficient RF energy must be supplied to the metal electrode to produce this lesion-forming temperature in the adjacent tissue for the desired duration.

Although preferred embodiments of this invention have been described above with a certain degree of particularity, those skilled in the art could make numerous alterations to the disclosed embodiments without departing from the spirit or scope of this invention. For example, the slotted openings can be of any width and any number, arranged in any variety of proximity from one to the next. The catheters of the present invention may also comprise a combination of slotted openings along the circumference of the catheter, perpendicular to the axis of the catheter, and long slits running along the axis of the catheter. Further, all directional references (e.g., upward, downward, outward, left, and right) are only used for identification purposes to aid the reader's understanding of the present invention, and do not create limitations, particularly as to the position, orientation, or use of the invention. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. An ablation catheter for ablating tissue, the ablation catheter comprising:
(a) a catheter shaft, said catheter shaft comprising a proximal portion and a distal portion, said distal portion being adapted to be inserted into a body having tissue to be treated and being disposed remotely from said proximal portion, said distal portion comprising a plurality of slotted openings each located on a circumference of the distal portion and being adapted to deliver a conductive medium to the tissue to be ablated, said plurality of slotted openings arranged along the circumference of the catheter shaft, wherein each slotted opening of said plurality of slotted openings spans at least about 25% of the circumference of said distal portion of said catheter shaft;
(b) a lumen disposed within said distal portion, said lumen adapted to carry the conductive medium; and
(c) an electrode disposed within said distal portion of the catheter shaft, said electrode being adapted to supply ablation energy to the conductive medium.

2. The ablation catheter of claim 1, further comprising a fluid manifold along at least a portion of the electrode.

3. The ablation catheter of claim 2 wherein said fluid manifold has tubing made of polyvinyl alcohol foam, expanded polytetrafluoroethylene, or a combination thereof.

4. The ablation catheter of claim 1, wherein said distal portion of said catheter shaft comprises at least one curved section.

5. The ablation catheter of claim 4, wherein said distal portion of said catheter shaft comprises at least one shape memory wire that is configured to create the at least one curved section.

6. The ablation catheter of claim 1, wherein each of said plurality of slotted openings spans about 33% of the circumference of said distal portion of said catheter shaft.

7. The ablation catheter of claim 1, wherein said distal portion of said catheter shaft comprises a plurality of curved sections to form a circular shape.

8. A catheter for treating tissue, the catheter comprising a catheter shaft, said catheter shaft comprising a proximal portion and a distal portion, said distal portion being adapted to be inserted into a body having tissue to be treated and being disposed remotely from said proximal portion, said distal portion comprising at least one slotted opening, with each of said at least one slotted opening being located on a circumference of the distal portion, wherein said at least one slotted opening is adapted to introduce a therapeutic energy, heat, fluid, or medicament to the tissue to be treated, wherein said at least one slotted opening is arranged perpendicular to the axis of said distal portion, and wherein said at least one slotted opening spans about one third to about two thirds of the circumference of the distal portion of the catheter shaft.

9. The catheter of claim 8, wherein said distal portion further comprises at least one lumen adapted to carry a conductive medium.

10. The catheter of claim 9 further comprising an electrode disposed within said distal portion and a conductive medium manifold running along a portion of said electrode, said conductive medium manifold having a plurality of passage ways through which a conductive medium may pass.

11. The catheter of claim 8 further comprising a lumen extending from said proximal portion to said distal portion, said lumen being adapted to carry a conductive medium from said proximal portion to said at least one slotted opening; and a metal electrode mounted within said lumen, wherein said metal electrode is adapted to supply ablation energy to the conductive medium.

12. The catheter of claim 11, wherein said distal portion of said catheter shaft comprises at least one curved section.

13. The ablation catheter of claim 12, wherein said ablation catheter further comprises a shape memory wire.

14. The ablation catheter of claim 13, wherein said catheter shaft further comprises a second lumen extending along said distal portion, and wherein said shape memory wire is located within said second lumen.

15. The ablation catheter of claim 11, wherein said metal electrode is a platinum flat wire adapted to be connected to an RF generator by an electrical lead that extends through at least a portion of the distal portion of said catheter shaft.

16. A method for treating cardiac arrhythmia, said method comprising
(a) inserting an ablation catheter into a patient having cardiac tissue to be treated; said ablation catheter comprising
i. a proximal portion and a distal portion, said distal portion being disposed remotely from said proximal portion, said distal portion comprising a plurality of slotted openings, wherein said plurality of slotted openings is adapted to introduce ablative energy to the cardiac tissue to be treated, wherein said plurality of slotted openings are each located on a circumference of said distal portion and wherein each slotted opening of said plurality of slotted openings spans between about 90° and about 2700 of the circumference of said catheter shaft;
ii. an electrode disposed within said distal portion, said electrode having a fluid manifold along at least a portion of said electrode, said electrode adapted to be connected to an ablative energy source;
wherein the plurality of slotted openings permit the catheter to ablate tissue in both the posterior wall of the left atrium and the pulmonary vein;
(b) placing the ablation catheter along the cardiac tissue to be treated; and
(c) applying ablative energy to the ablation catheter to form lesions on the cardiac tissue.

17. An ablation catheter for ablating tissue, the ablation catheter comprising
(a) a catheter shaft, said catheter shaft comprising a proximal portion and a distal portion, said distal portion being adapted to be inserted into a body having tissue to be treated and being disposed remotely from said proximal portion, said distal portion comprising a plurality of slotted openings each located on a circumference of the distal portion, said plurality of slotted openings arranged along the circumference of the catheter shaft, wherein each slotted opening of said plurality of slotted openings spans at least about 90° of the circumference of said distal portion of said catheter shaft; and
(b) a metal electrode disposed within said distal portion of the catheter shaft, said electrode being adapted to supply ablation energy through the slotted openings to the tissue to be ablated.

18. The ablation catheter of claim 17, further comprising a fluid manifold along at least a portion of the metal electrode and wherein said slotted openings are adapted to deliver conductive fluid to the tissue to be ablated.

19. The ablation catheter of claim 18 wherein said fluid manifold has tubing made of a porous polymer.

20. The ablation catheter of claim 17 wherein the slotted opening span about 33% to about 67% of the circumference of said distal portion.

21. A method for simultaneously ablating tissue that is in at least two different orientations within a body, the method comprising:
(a) inserting an ablation catheter into a patient having tissue to be ablated; said ablation catheter comprising
i. a proximal portion and a distal portion, said distal portion being disposed remotely from said proximal portion, said distal portion comprising a plurality of slotted openings, wherein said plurality of slotted openings is adapted to introduce ablative energy to the cardiac tissue to be treated, wherein said plurality of slotted openings are located on a circumference of said distal portion and wherein each slotted opening of said plurality of slotted openings spans between about 33% and about 67% of the circumference of said catheter shaft;
ii. an electrode disposed within said distal portion, said electrode adapted to be connected to an ablative energy source;
(b) placing the ablation catheter along the tissue to be treated; and
(c) applying ablative energy to the ablation catheter to form lesions simultaneously on tissue that is in at least two different orientations.
